(19) United States
(12) Patent Application Publication
(10) Pub. No.: US 2025/0173869 A1
(43) Pub. Date: May 29, 2025

(54) METHOD FOR AIDING THE DIAGNOSE OF SPINE CONDITIONS

(71) Applicant: CAERUS MEDICAL, Loos (FR)

(72) Inventors: Naïm JALAL, Loos (FR); Eric CHEVALIER, Loos (FR); David VITTECOQ, Loos (FR)

(73) Assignee: CAERUS MEDICAL, Loos (FR)

(21) Appl. No.: 18/841,417

(22) PCT Filed: Mar. 10, 2023
(86) PCT No.: PCT/EP2023/056219

§ 371 Date: Aug. 26, 2024

Related U.S. Application Data
(63) Continuation of application No. 17/692,605, filed on Mar. 11, 2022, now abandoned.

(30) Foreign Application Priority Data
Mar. 11, 2022 (EP) 22305288.7

(51) Int. Cl. G06T 7/00; G06T 7/62

(52) U.S. Cl. CPC G06T 7/0014; G06T 7/62; G06T 2207/20084; G06T 2207/30012

(57) ABSTRACT

A computer-implemented method for providing diagnosis aid for diagnosing spinal condition by providing feature(s) of a subject's spine. The method includes: a receiving step, receiving subject-associated imaging signals, the imaging signals representing at least a part of the subject's spine; a first processing step, computing a first set of spine data based on a first imaging signal; a first prediction step, computing a first output relating to a first anatomical structure of the subject's spine based on the first set of spine data; a second processing step, computing a second set of spine databased on a second imaging signal; and a second prediction step, computing a second output relating to a second anatomical structure of the subject's spine based on the second set of spine data and the first output, the second output including a second feature of the subject's spine representing at least one spinal condition.

### FIELD OF INVENTION

The invention concerns a computer-implemented method for providing diagnosis aid for diagnosing spinal condition in a subject's spine by providing at least one feature of the subject's spine.

The invention also concerns a system for providing diagnosis aid for diagnosing spinal condition in a subject's spine by providing at least one feature of the subject's spine implementing the above-cited method.

The invention relates to the field of medical images analysis, and more specifically analysis for identifying spine diseases.

### BACKGROUND OF INVENTION

Low back pain constitutes one of the major causes of disability worldwide. Low back pain can be due to specific causes (such as tumors, infections or osteoporosis) or to non-specific causes.

Degenerative disc diseases are the most frequent underlying conditions associated with non-specific low back pain. For instance, it is estimated that the number of patients suffering from lumbar degenerative disc disease reaches 266 million each year worldwide.

Patients with persistent low back pain experience unnecessary extended pain, anxiety, and poor quality of life. Moreover, the economic burden associated to such conditions cannot be neglected: for instance, in the USA, the expenses associated to low back pain have gone from USD 26.3 billion dollars in 1998 to more than 100 billion dollars in 2011.

In clinical practice, magnetic resonance imaging (MRI) is the gold standard for diagnosing low back pain and particularly degenerative disc diseases. More precisely, several imaging sequences (such as, at least, sagittal T1w and T2w MRI, and axial T2w MRI) are used by a clinician to diagnose one or several conditions among a very wide range of conditions (discs and endplate degeneration, herniated disc, spondylolisthesis, laterolisthesis, stenosis of the central and lateral canal, vertebral collapse, etc.). This analysis requires both significant time and expertise of the spine that common radiologists do not have. As a consequence, the reliability of these diagnoses is moderate to barely substantial.

Therefore, it is crucial to provide a tool that allows for better early low back pain diagnosis in order to improve both patient outcome and economic and societal costs.

A platform has been recently developed, with the aim of offering diagnosis aid of spinal conditions based on MRI. More precisely, such platform is configured to segment acquired MRI images, and to output a diagnosis based on measurements performed on the computed segmented MRI images.

However, such platform is not satisfactory.

Indeed, resorting to MRI does not give access to reliable diagnosis of hypo/hyperlordosis, scoliosis and spondylolisthesis with the standard Meyerding scale. This is due to the fact that MRI images are acquired without load on the spine; in other words, during MRI, the spine is in a lengthened state. Consequently, the relevancy of such platform for detecting the aforementioned conditions is moot. Furthermore, such platform is unable to detect several conditions, for example disc degeneration, plateaus degeneration, Schmorl nodes, vertebral compression or even facet arthritis, so that a clinician can only partially rely on the outputs of this platform.

For instance, patent application U.S. 62/882,076 uses a 3-step approach consisting of: (1) a segmentation phase allowing the identification of the area of interest, (2) a categorization and evaluation of a set of anatomical or pathological measurements, and (3) a diagnosis. This approach is insufficient because it only relies on measurements and does not take into account the intricacies between several pathologies.

Furthermore, segmentation of the acquired MRI may be ill-done, thereby leading to an unreliable diagnosis output by such platform.

A purpose of the invention is to provide a device for determining at least one feature of a subject's spine, said device being more versatile and reliable than the aforementioned platform.

### SUMMARY

A first aspect of the invention relates to a computer-implemented method for providing diagnosis aid for diagnosing spinal condition in a subject's spine by providing at least one feature of the subject's spine, the method comprises:
a receiving step wherein at least a first and a second imaging signals associated to the subject are received, the at least first and/or second imaging signals being representative of at least a part of the subject's spine;
a first processing step wherein a first set of spine data is computed based on the at least first imaging signal;
a first prediction step, wherein at least one first output relating to a first anatomical structure of the subject's spine is computed based on the first set of spine data, the at least one first output including a first feature of the subject's spine;
a second processing step wherein a second set of spine data is computed based on the at least second imaging signal;
a second prediction step, wherein at least one second output relating to a second anatomical structure of the subject's spine is computed based on the second set of spine data and on the at least one first output, the at least second output including a second feature of the subject's spine representative of at least one spinal condition.

Such method has a lot of advantages. It enables to do a reliable diagnosis of at least one spine condition by outputting information, such as the first and second features for example, that take into account:
the intricacies between the different anatomical structures of the spine, the at least second output relating to a second anatomical structure of the subject's spine being computed based on the second set of spine data and on the at least one first output relating to a first anatomical structure; as well as
the intricacies between several pathologies, where the first and second features may be related to a first and a second condition in the subject's spine.

According to other advantageous aspects of the invention, the method includes one or more of the following features, taken alone or in any technically possible combination:
the first processing step and/or the second processing step comprise a pre-processing step computing images from the at least first and/or second imaging signals, preferably the computed images are successive slices of at least a first and/or a second part of the subject's spine representing a 3D view of the at least first and/or second part of the subject's spine;
the pre-processing step also computes on the images at least one of: a resizing, an intensity transformation, an artefact correction, a bias correction, and/or a pixel intensity distribution normalization;
the pre-processing step is followed by an anatomical structure segmentation step which processes the images in order to identify boundaries of predetermined anatomical structures;
the anatomical structure segmentation step is implemented by a deep learning model trained on a library of spine images of reference, the deep learning model outputting a set of raw 2D segmentation masks, each mask being associated to a corresponding anatomical structure appearing respectively in the image pre-processed, the deep learning model being preferably a convolutional neural network;
the anatomical structure segmentation step is followed by:
a post-processing step on each set of raw 2D segmentation masks so as to eliminate false positives and artefacts, and
a labelling step wherein the predetermined anatomical structures identified in the segmented images are labelled;
the predetermined anatomical structures include vertebrae and/or intervertebral discs and, for each vertebra segmented in the images obtained after the anatomical segmentation step a vertebra centroid is computed, preferably the vertebrae centroid is computed as the center of mass of said segmented vertebra based on the corresponding segmentation masks and, respectively for each intervertebral discs in the images obtained after the anatomical segmentation step an intervertebral disc centroid is computed, preferably the intervertebral disc centroid is computed as the center of mass of said segmented intervertebral disc;
the at least one first output is data or parameter resulting of a calculation performed on the first set of spine data, such as a grade (number), a landmark (vector), an area (matrix), a volume (tensor), a segmented result, a position, a probability, or weights of an artificial intelligence model determined during or after its training for performing the calculation, like a neural network;
the first feature includes first condition data indicative of an occurrence of a predetermined first condition in the first anatomical structure;
the first condition is one of: Modic type endplate changes, Schmorl node, anterolisthesis, retrolisthesis, laterolisthesis, disc degeneration, hypolordosis, hyperlordosis, scoliosis, disc herniation (symmetric bulging, asymmetric bulging protrusion and extrusion) and its location, sequestration status, nerve root compression status, spinal canal stenosis and its origins, lateral recess stenosis and its origins, neural foraminal stenosis and its origins, compression fracture and its acute status, paraspinal muscle atrophy, fatty involution in paraspinal muscle, facet arthropathy and its origins, tumors, infection, pain, spondylosis;
the second feature includes second condition data indicative of an occurrence of a predetermined second condition in the second anatomical structure;
the occurrence of the second condition is related to the occurrence of the first condition;
the second anatomical structure is the first anatomical structure;
the second anatomical structure is distinct from the first anatomical structure, the second anatomical structure being adjacent to the first anatomical structure or located at a distance from the first anatomical structure;
the at least one first output is representative of a relationship between the first feature and the second feature;
the first condition data is indicative of an occurrence of Modic type endplate changes and the second condition is disc degeneration;
the first and second prediction steps are respectively implemented by a first and a second neural networks, the first neural network being trained to generate the at least one first output relating to the first anatomical structure of the subject's spine based on the first set of spine data, and the second neural network being trained to generate the at least one second output relating to the second anatomical structure of the subject's spine based on the second set of spine data and the at least one first output, wherein the at least one first output is related to weights at specific depth levels of the first neural network;
the first prediction step is configured to predict the existence of a herniated disc, and the second prediction step is configured to predict the existence of a spinal canal stenosis; and/or
the first prediction step computes first features that include information relating to the occurrence of a given condition, as well as a hint for this condition, and the second prediction step is configured to compute the at least one second output based on the second set of spine data and on the at least one first output comprising the first features and the hint.

Another aspect of the present invention relates to a system for providing diagnosis aid for diagnosing spinal condition in a subject's spine by providing at least one feature of the subject's spine, the system being configured to implement a method as previously described, the system comprises:
a processing device configured to receive at least a first and a second imaging signals associated to the subject, the at least first and/or second imaging signals being representative of at least a part of the subject's spine; and to compute a first and a second set of spine data respectively based on the at least first and second imaging signals;
a prediction device configured to compute at least one first output relating to a first anatomical structure of the subject's spine based on the first set of spine data, the at least first output including a first feature of the subject's spine, and to compute at least one second output relating to a second anatomical structure of the subject's spine based on the second set of spine data and on the at least one first output, the at least second output including a second feature of the subject's spine representative of at least one spinal condition.

The method is designed to be performed on a distant server, such as a cloud computing platform, that is accessed remotely by users over a network. In one embodiment, the method comprises receiving input data from a user, processing the input data using one or more algorithms to generate an output, and transmitting the output back to the user over the network. The method may be implemented using any suitable programming language or framework, and may utilize various types of hardware and software resources available on the distant server. By executing the method on a cloud computing platform, users can benefit from the scalability, reliability, and cost-effectiveness of cloud-based computing resources, while avoiding the need to maintain their own expensive hardware and software infrastructure.

Another aspect of the present disclosure relates to a device for determining at least on feature of a subject's spine, the device including a processing unit comprising:
at least one first module configured to receive a corresponding first set of spine image data associated to the subject, the first module being further configured to compute, based on spine image data representative of at least part of an examined spine, at least one first output relating to a first anatomical structure of the examined spine, the at least one first output including a first feature of the examined spine;
at least one second module, distinct from the first module, configured to receive:
   at least one first output of the first module; and
   a corresponding second set of spine image data associated to the subject,
the second module being further configured to compute, based on spine image data representative of at least part of the examined spine and the at least one first output of the first module, at least one second output relating to a second anatomical structure of the examined spine, the at least one second output including a second feature of the examined spine.

Indeed, regarding prediction of conditions, by using first outputs of the first module to compute second outputs (such as the second feature) of the second module, the present disclosure allows to take into account the fact that knowledge relative to a given anatomical structure of the spine has consequences either on other anatomical structures, or on conditions of the same anatomical structure. This is due to the biomechanical correlation and compensation mechanisms across the length of the spine.

Furthermore, regarding processing of imaging signals, by using first outputs of the first module to compute second outputs (such as the second feature) of the second module, the present disclosure allows to combine different acquisition sequences, for instance of a same anatomical structure of the spine, to achieve segmentation that is more reliable than the segmentation results that would be obtained without performing the approach according to the invention.

According to other advantageous aspects of the present disclosure, the device includes one or more of the following features, taken alone or in any technically possible combination:
each first output received by the second module is representative of a relationship between the first feature computed by the first module and the second feature computed by the second module;
the first module is configured to implement a first artificial intelligence model to compute the first feature, and the second module is configured to implement a second artificial intelligence model to compute the second feature, the first artificial intelligence model and the second artificial intelligence model being configured to provide, during training, at least one weight of the first artificial intelligence model that is relevant for computation of the second feature by the second artificial intelligence model, the at least one first output received by the second module including each provided weight;
the first feature includes first condition data indicative of an occurrence of a predetermined first condition in the first anatomical structure, and the second feature includes second condition data indicative of an occurrence of a predetermined second condition in the second anatomical structure, the occurrence of the second condition being related to the occurrence of the first condition;
the second anatomical structure is the first anatomical structure, the occurrence of the second condition in the first anatomical structure being related to the occurrence of the first condition in the first anatomical structure;
at least part of the first spine image data and/or the second spine image data is representative of a geometry of the first anatomical structure and/or the second anatomical structure;
the first spine image data and/or the second spine image data comprise a corresponding label, a corresponding location, a location of at least one respective landmark, a corresponding boundary, a corresponding bounding box and/or a corresponding measurement;
the measurement includes a distance, an area, a volume and/or an angle within the first anatomical structure and/or the second anatomical structure, and/or a distance, an area, a volume and/or an angle between landmarks of the first anatomical structure and/or the second anatomical structure;
at least one of the first feature and the second feature is representative of a geometry of the first anatomical structure and/or the second anatomical structure;
at least one of the first feature and the second feature comprises a corresponding label, a corresponding location, a location of at least one respective landmark, a corresponding boundary, a corresponding bounding box and/or a corresponding measurement;
the measurement includes a distance, an area, a volume and/or an angle within the first anatomical structure and/or the second anatomical structure, and/or a distance, an area, a volume and/or an angle between landmarks of the first anatomical structure and/or the second anatomical structure;
the first set of spine image data includes first data acquired according to a first acquisition sequence, and the second set of spine image data includes second data acquired according to a second acquisition sequence distinct from the first acquisition sequence, the second anatomical structure being the first anatomical structure;
the first data and the second data include imaging signals representative of the first anatomical structure and/or the second anatomical structure;
the imaging signals comprise images.

The present disclosure also relates to a system for detecting at least one condition in a subject's spine, the system comprising:
a first device as defined above, the first set of spine image data and the second set of spine image data including at least one imaging signal representative of the subject's spine, the first feature being representative of a geometry of the first anatomical structure of the subject's spine, and the second feature being representative of a geometry of the second anatomical structure of the subject's spine; and/or
a second device as defined above, the first set of spine image data and the second set of spine image data including at least one feature representative of a geometry of at least one of the first anatomical structure of the subject's spine and/or the second anatomical structure of the subject's spine, the first feature being representative of the occurrence of a first condition in the first anatomical structure, and the second feature being representative of a second condition in the second anatomical structure.

According to other advantageous aspects of the present disclosure, the system includes one or more of the following features, taken alone or in any technically possible combination:
at least part of the first set of spine image data and the second set of spine image data received by the second device is an output of the first device;
the first condition and/or the second condition is a lumbar pathology;
the first condition and/or the second condition is a grade on a predetermined scale representative of at least one corresponding spine pathology, such as a lumbar pathology, preferably a grade on the Pfirrmann scale and/or a grade on the Modic type endplate changes scale;
the first set of spine image data and/or the second set of spine image data of the first device includes at least one of: an X-ray radiography imaging signal, a magnetic resonance imaging signal, and an ultrasound signal.

The present disclosure also relates to a computer-implemented method for determining at least one feature of a subject's spine, the method comprising:
to at least one first artificial intelligence model, providing a first set of spine image data associated to the subject, the first artificial intelligence model being configured to compute, based on spine image data representative of at least part of an examined spine, at least one first output relating to a first anatomical structure of the examined spine, the at least one first output including a first feature of the examined spine;
to at least one second artificial intelligence model, distinct from the first artificial intelligence model, providing:
   at least one first output of at least one first artificial intelligence model; and
   a corresponding second set of spine image data associated to the subject,
the second artificial intelligence model being further configured to compute, based on spine image data representative of at least part of the examined spine and the at least one first output of the second artificial intelligence model, at least one second output relating to a second anatomical structure of the examined spine, the at least one second output including a second feature of the examined spine.

According to other advantageous aspects of the present disclosure, the method includes one or more of the following features, taken alone or in any technically possible combination:
each first output received by the second artificial intelligence model is representative of a relationship between the first feature computed by the first artificial intelligence model and the second feature computed by the second artificial intelligence model;
the method includes a step of training the first artificial intelligence model and the second artificial intelligence model to provide at least one weight of the first artificial intelligence model that is relevant for computation of the second feature by the second artificial intelligence model, the at least one first output received by the second artificial intelligence model including each provided weight;
at least one of the first feature and the second feature is representative of a geometry of the first anatomical structure and/or the second anatomical structure;
at least one of the first feature and the second feature comprises a corresponding label, a corresponding location, a location of at least one respective landmark, a corresponding boundary, a corresponding bounding box and/or a corresponding measurement;
the measurement includes a distance, an area, a volume and/or an angle within the first anatomical structure and/or the second anatomical structure, and/or a distance, an area, a volume and/or an angle between landmarks of the first anatomical structure and/or the second anatomical structure;
the first set of spine image data includes first data acquired according to a first acquisition sequence, and the second set of spine image data includes second data acquired according to a second acquisition sequence distinct from the first acquisition sequence, the second anatomical structure being the first anatomical structure;
the first data and the second data include imaging signals representative of the first anatomical structure and/or the second anatomical structure;
the imaging signals comprise images.

According to further advantageous aspects of the present disclosure, the method may also include one or more of the following features, taken alone or in any technically possible combination (and/or in combination with the aforementioned features):
the first feature includes first condition data indicative of an occurrence of a predetermined first condition in the first anatomical structure, and the second feature includes second condition data indicative of an occurrence of a predetermined second condition in the second anatomical structure, the occurrence of the second condition being related to the occurrence of the first condition;
the second anatomical structure is the first anatomical structure, the occurrence of the second condition in the first anatomical structure being related to the occurrence of the first condition in the first anatomical structure;
at least part of the first spine image data and/or the second spine image data is representative of a geometry of the first anatomical structure and/or the second anatomical structure;
the first spine image data and/or the second spine image data comprise a corresponding label, a corresponding location, a location of at least one respective landmark, a corresponding boundary, a corresponding bounding box and/or a corresponding measurement;
the measurement includes a distance, an area, a volume and/or an angle within the first anatomical structure and/or the second anatomical structure, and/or a distance, an area, a volume and/or an angle between landmarks of the first anatomical structure and/or the second anatomical structure.

The present disclosure also relates to a computer program product comprising instructions which, when the program is executed by a computer, cause the computer to automatically carry out the steps of a method for providing diagnosis aid for diagnosing spinal condition in a subject's spine by providing at least one feature of the subject's spine according to any one of the embodiments described hereabove (i.e. the instructions may cause the computer to carry out the steps of the method according to a single one of those embodiments, or any two or more of those embodiments). The computer program product may be implemented on a distant server, such as a cloud computing platform, that is accessed remotely by users over a network.

The present disclosure also relates to a non-transitory computer readable storage medium comprising instructions which, when the program is executed by a computer, cause the computer to carry out the steps of the method according to any one of the embodiments described hereabove.

Such a non-transitory program storage device can be, without limitation, an electronic, magnetic, optical, electromagnetic, infrared, or semiconductor device, or any suitable combination of the foregoing. It is to be appreciated that the following, while providing more specific examples, is merely an illustrative and not exhaustive listing as readily appreciated by one of ordinary skill in the art: a portable computer diskette, a hard disk, a ROM, an EPROM (Erasable Programmable ROM) or a Flash memory, a portable CD-ROM (Compact-Disc ROM). Furthermore, the non-transitory computer readable storage medium may be implemented on a distant server, such as a cloud computing platform, that is accessed remotely by users over a network.

### BRIEF DESCRIPTION OF THE DRAWINGS

The invention will be better understood with the attached figures, in which:

FIG. 1 is a schematic representation of a first embodiment of a computer-implemented method for providing aid for diagnosing spinal condition in a subject's spine;

FIG. 2 is a schematic representation of a first embodiment of a spine condition detection system according to the present disclosure;

FIG. 3 is an example illustrating operation of the spine condition detection system of FIG. 2;

FIG. 4 is a schematic representation of a second embodiment of a spine condition detection system according to the present disclosure; and

FIG. 5 is a schematic representation of a third embodiment of a spine condition detection system according to the present disclosure.

### DEFINITIONS

According to the invention, the expression “anatomical structure” refers to a biological structure of the spinal region that is distinguished from neighboring structures. Anatomical structures may include vertebrae, ligaments, tendons, spinal cord, nerve roots, pedicle, neural foramina, intervertebral discs, facets, facet joints (or synovial joints), joint capsules, paraspinal muscles, spinal segments, lumbar spine, thoracic spine, cervical spine, or parts thereof.

According to the present invention, the expression “geometry of an anatomical structure” refers to a corresponding label, a corresponding location, a location of at least one respective landmark, a corresponding boundary, a corresponding bounding box and/or a corresponding measurement, wherein the measurement preferably includes a distance, an area, a volume and/or an angle within the anatomical structure, and/or a distance, an area, a volume and/or an angle between landmarks of the anatomical structure.

According to the present invention, the expression “imaging signal” refers to a signal output by a medical imaging device and that is representative of at least one imaged anatomical structure. The imaging signal may comprise a time-domain signal, a spectral-domain signal, and/or an image of said at least one imaged anatomical structure. The imaging signal may also comprise clinical data relative to the person being examined (e.g. age, sex, size, weight, activity, previous or current pathological condition(s), etc.), as well as information about the medical imaging device (e.g. focal distance, size of the field of view, resolution, contrast, orientation/anatomic plane imaged . . . ).

According to the invention, the expression “condition” refers to a state of the spine, involving one or several anatomical structure(s) of the spine, either adjacent or not. Said condition may be a pathology, and/or disease, and/or injury of the spine.

According to the invention, the expression “processing unit” refers to a processing device, regardless of its implementation, capable of executing instructions to perform associated and/or resulting functionalities.

According to the invention, the expression “spine image data” refers to data determined based on an imaging of at least part of a subject's spine. The spine image data may include imaging signals (as previously defined) and/or information computed based on said imaging signals.

### DETAILED DESCRIPTION

The present description illustrates the principles of the present disclosure. It will thus be appreciated that those skilled in the art will be able to devise various arrangements that, although not explicitly described or shown herein, embody the principles of the disclosure and are included within its spirit and scope.

All examples and conditional language recited herein are intended for educational purposes to aid the reader in understanding the principles of the disclosure and the concepts contributed by the inventor to furthering the art, and are to be construed as being without limitation to such specifically recited examples and conditions.

Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that the block diagrams presented herein may represent conceptual views of illustrative circuitry embodying the principles of the disclosure. Similarly, it will be appreciated that any flow charts, flow diagrams, and the like represent various processes which may be substantially represented in computer readable media and so executed by a computer or processor, whether or not such computer or processor is explicitly shown.

The functions of the various elements shown in the figures may be provided through the use of dedicated hardware as well as hardware capable of executing software in association with appropriate software. When provided by a processor, the functions may be provided by a single dedicated processor, a single shared processor, or a plurality of individual processors, some of which may be shared.

It should be understood that the elements shown in the figures may be implemented in various forms of hardware, software or combinations thereof. Preferably, these elements are implemented in a combination of hardware and software on one or more appropriately programmed general-purpose devices, which may include a processor, memory and input/output interfaces.

### First Method Embodiment

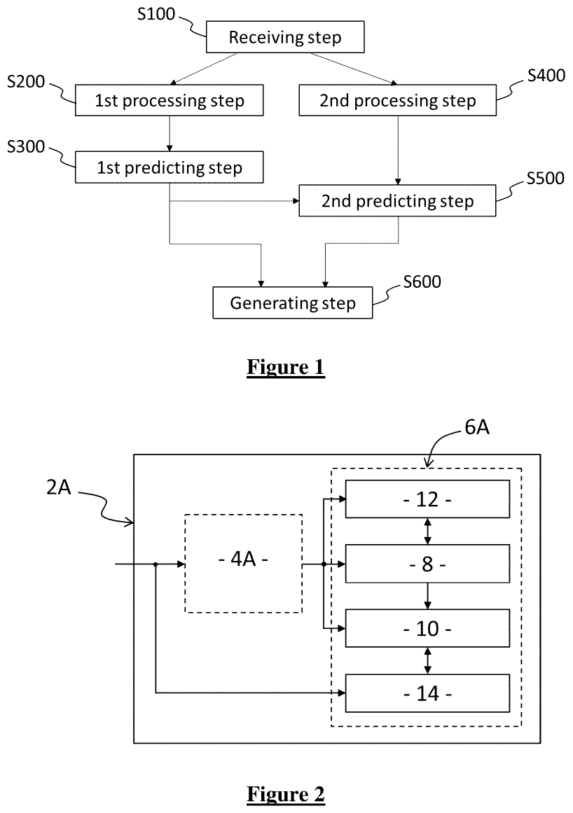

FIG. 1 illustrates a first embodiment of a computer-implemented method for providing diagnosis aid for diagnosing spinal condition in a subject's spine by providing at least one feature of the subject's spine.

The method comprises:
a receiving step S100, wherein at least a first and a second imaging signals associated to the subject are received, the at least first and/or second imaging signals being representative of at least a part of the subject's spine;
a first processing step S200, wherein a first set of spine data is computed based on the at least first imaging signal;
a first prediction step S300, wherein at least one first output relating to a first anatomical structure of the subject's spine is computed based on the first set of spine data, the at least one first output including a first feature of the subject's spine;
a second processing step S400, wherein a second set of spine data is computed based on the at least second imaging signal;
a second prediction step S500, wherein at least one second output relating to a second anatomical structure of the subject's spine is computed based on the second set of spine data and on the at least one first output, the at least second output including a second feature of the subject's spine representative of at least one spinal condition.

Advantageously, the method may also comprise a generating step S600, wherein a medical report is generated in order to aid the medical practitioner, in charge of the subject, diagnose the presence of at least one condition in the subject's spine. If there is at least one condition detected or not, the medical report will be a support to explain to the subject where and what condition it is or is not.

The medical report may be completely customizable. For example, the medical report may comprise some of the images processed during the first S200 and second S400 processing steps to illustrated the localization of the condition and the measurements realized to detect the condition, as well as information relatives to the first S300 and second S500 prediction steps such as the first and second features.

### The Receiving Step S100

Advantageously, the at least first and/or second imaging signals may comprise images coming from medical imaging devices such as X-Ray or ultrasound apparatuses, MRI or CT scans, for example but not restricted to this list. In some cases, the at least first and/or second imaging signals may comprise images of the same medical imaging device, acquired during the same imaging sequence or from different sequences, having the same modality (e.g. T1, T1w, T2, T2w, STIR, FLAIR, 3D, Dixon and so on) or different modalities. In other cases, the first and/or second imaging signals may comprise images of different medical apparatuses. For example, the at least first and/or second imaging signals may comprise a combination of different modalities (e.g. T1w and T2w, X-ray and ultrasound . . . ), as well as images having the same anatomical plane or different anatomical planes between sagittal, axial, coronal planes.

Preferably, the images coming from medical imaging devices are 2D images, but they may also be 3D images comprising successive slices of 2D images, especially for the diagnosis of in-depth pathologies.

Furthermore, the at least first and/or second imaging signals may comprise clinical data with or without images from the medical imaging device(s). These clinical data may be relative to the subject being examined (e.g. age, sex, size, weight, activity, previous or current pathological condition(s), etc.), as well as information about the medical imaging device(s) used to acquire the at least first and second imaging signals (e.g. focal distance, size of the field of view, resolution, contrast, orientation/anatomic plane imaged . . . ).

The at least first and second imaging signals may also be identical. Advantageously, in this case, some processing can be mutualized which makes it possible to reduce the processing time, particularly during the first S200 and second S400 processing steps.

Thus, compare to prior art methods that use only one type of view or only one modality, the present method enables to diagnose a spinal condition based on a combination of views (sagittal, axial, and/or coronal), modalities (T1, T1w, T2, T2w, STIR, FLAIR, 3D, and/or Dixon . . . ) and clinical data, enabling a more precise and reliable diagnosis.

### The First S200 and Second S400 Processing Steps

In the case where the at least first and second imaging signals are identical, the first and second sets of spine data computed by the first S200 and second S400 processing steps may be identical and/or have mutualized sub-steps.

Whether the first and second sets of spine data are identical or not, computing them in the first S200 and/or second S400 processing steps may comprise one or more sub-steps which may be applied separately, in combination or successively such as: a pre-processing step, an anatomical structure segmentation, a post-processing step, a centroid computation, an intervertebral disc position computation, a labelling step.

The pre-processing step computes images from the at least first and/or second imaging signals. Preferably, the computed images are successive slices of at least a first and/or a second part of the subject's spine, representing a 3D view of the at least first and/or second part of the subject's spine.

Furthermore, the pre-processing step may also compute on the images at least one of: a resizing, an intensity transformation, an artefact correction, a bias correction and/or a pixel intensity distribution normalization.

The anatomical structure segmentation step is generally realized on the pre-processed images in order to identify boundaries of predetermined anatomical structures. As previously described, the predetermined anatomical structures may include vertebrae, ligaments, tendons, spinal cord, nerve roots, pedicle, neural foramina, intervertebral discs, facets, facet joints (or synovial joints), joint capsules, paraspinal muscles, spinal segments, lumbar spine, thoracic spine, cervical spine, or parts thereof.

The anatomical structure segmentation step may be implemented by a deep learning model such as convolutional neural network. To do so, the deep learning model is trained on a library of spine images of reference wherein the predetermined anatomical structures have been outlined by medical practitioners or wherein the segmented images are validated by medical practitioner during the training.

Advantageously, the deep learning model may be a collection of different models (it can be models with different architectures, or similar architecture but different weights) that may or may not be trained on the same training data. The segmentation made by the ensemble models can then be combined through voting statistics, or by a more sophisticated methods that learns how much to trust each model and under what conditions.

Ensemble models have the advantage over single models to: increase performance and better segmentations, have more robustness through the spread of segmentations and the voting of each model of the ensemble.

For example, the discs can be identified directly by segmentation models, object detection models but also extrapolated from the vertebrae segmentation. Each of these approaches have their strengths and weaknesses depending on the case. By combining all these pipelines, more robust and performant models in real-life applications are ensured.

As previously described, the method and thus the deep learning model may allow to analyze different views and modalities. For example, but not restricted to, the deep learning model may be trained on the following types of images: Sagittal T1w MRI Sequences in DICOM Format, Sagittal T2w MRI Sequences in DICOM Format, Sagittal T2 STIR MRI Sequences in DICOM Format, Axial T2w MRI Sequences in DICOM Format, Coronal T1w MRI Sequences in DICOM Format, Coronal T2 STIR MRI Sequences in DICOM Format, Sagittal DIXON MRI Sequences in DICOM Format, and/or 3D T2w MRI Sequences in DICOM Format.

Advantageously, the deep learning model, preferably the convolutional neural network, outputs a set of raw 2D segmentation masks, each mask being associated to a corresponding anatomical structure appearing respectively in the image(s) pre-processed.

Thus, the outputs of the anatomical structure segmentation step are the crops of the areas of interest, i.e. the areas around the discs and vertebrae, wherein the vertebrae can be labelled, i.e. the anatomical level of each vertebra.

So as to eliminate the false positives and artefacts, a segmentation post-processing step may be applied on each set of raw 2D segmentation masks. An effective way to do so is to compare the successive segmented images obtained after the anatomical structure segmentation step, when the at least first and/or second imaging signals comprise successive slices of at least a first and/or a second part of the subject's spine. This allows to identify common structures that appears in multiple slices and delete noise/artefacts present in 2D slices.

When the predetermined anatomical structures include vertebrae and/or intervertebral discs, for each vertebra segmented in the images obtained after the anatomical segmentation step, a vertebra centroid and/or an intervertebral disc centroid may be computed. Preferably, the vertebrae centroid may be computed as the center of mass of said segmented vertebra identified in the segmented images such as in the corresponding segmentation masks. Respectively, the intervertebral disc centroid may be computed as the center of mass of said segmented intervertebral disc identified in the segmented images.

Another way to determine the intervertebral disc centroid is to do a polynomial regression of the spinal curve using the vertebras centroids as anchor points. The polynomial degree may be defined as the number of anchor points identified. This allows to interpolate the intervertebral discs (IVDs) centroids as the points on the spinal curve that are mid-distance between two vertebrae centroids.

The labelling step consists in labelling the predetermined anatomical structures identified in the images obtained after the anatomic structure segmentation step. The labelling step is generally applied after the segmentation post-processing step for a better labelling but it can also be applied at the end of the anatomical structure segmentation step. For example, each computed vertebra centroid is assigned to the corresponding vertebra, and respectively each computed intervertebral disc centroid is assigned to the corresponding intervertebral disc.

Furthermore, bounding boxes may be computed on the segmented images and around area/anatomical structures of interest such as the intervertebral discs. A way to compute the bounding boxes around intervertebral discs is to rotate each labelled mask with respect to the spinal curve angle, and for each intervertebral disc of interest, the surrounding vertebrae are considered, and their centers are rotated as well with respect to the spinal curve angle. The intervertebral disc bounding boxes are then computed by cropping the rotated labelled mask by using the rotated centers coordinates of the surrounding vertebrae and an offset. The distance between the intervertebral disc and its neighboring vertebrae may be used to compute the height of the bounding box, so that each bounding box represent a region of interest including said anatomical structure. The same process can be done to compute bounding boxes around each vertebra.

### The First S300 and Second S500 Prediction Steps

Advantageously, the at least one first output computed in the first prediction step S300 is data or parameter(s) resulting of a calculation performed on the first set of spine data, such as a grade (number), a landmark (vector), an area (matrix), a volume (tensor), a segmented result, a position, a probability, or weights of an artificial intelligence model determined during or after its training for performing the calculation, like a neural network.

The first feature may include first condition data indicative of an occurrence of a predetermined first condition in the first anatomical structure.

For example, the first condition may be one of: Modic type endplate changes, Schmorl node, anterolisthesis, retrolisthesis, laterolisthesis, disc degeneration, hypolordosis, hyperlordosis, scoliosis, disc herniation (symmetric bulging, asymmetric bulging protrusion and extrusion) and its location, sequestration status, nerve root compression status, spinal canal stenosis and its origins, lateral recess stenosis and its origins, neural foraminal stenosis and its origins, compression fracture and its acute status, paraspinal muscle atrophy, fatty involution in paraspinal muscle, facet arthropathy and its origins, tumors, infection, pain, spondylosis.

In the same manner, the second feature may include second condition data indicative of an occurrence of a predetermined second condition in the second anatomical structure.

Advantageously, the occurrence of the second condition may be related to the occurrence of the first condition.

In some cases, the second anatomical structure is the first anatomical structure, and in others cases the second anatomical structure may be distinct from the first anatomical structure, the second anatomical structure being adjacent to the first anatomical structure or located at a distance from the first anatomical structure.

Furthermore, the at least one first output may advantageously be representative of a relationship between the first feature and the second feature.

For example, the first condition data may be indicative of an occurrence of Modic type endplate changes and the second condition of disc degeneration.

Thus, the outputs of the first S300 and second S500 prediction steps may be the identification of the pathologies present as well as the evaluation of their grades according to a reference specific to each pathology, for each anatomical level present in the image and detected by the previous processing steps S200 and S400.

Advantageously, the first S300 and second S500 prediction steps are respectively implemented by a first and a second neural networks, the first neural network being trained to generate the at least one first output relating to the first anatomical structure of the subject's spine based on the first set of spine data, and the second neural network being trained to generate the at least one second output relating to the second anatomical structure of the subject's spine based on the second set of spine data and the at least one first output, wherein the at least one first output is related to weights at specific depth levels of the first neural network.

The training sets for the first and second neural networks may be the same set as the training set used for the deep learning models or just one or more libraries of images wherein a pathology/condition in the imaged spine has already been identified by a medical practitioner.

In another example, the first prediction step may be configured to predict the existence of a herniated disc, and the second prediction step may be configured to predict the existence of a spinal canal stenosis.

Advantageously, the first prediction step computes first features that include information relating to the occurrence of a given condition, as well as a hint for this condition, and the second prediction step is configured to compute the at least one second output based on the second set of spine data and on the at least one first output comprising the first features and the hint.

Table 1 illustrates an example of the types of imaging signals used and processed to predict some pathologies/conditions according to the present method, as well as relations and correlations possible between different pathologies and their predictions.

TABLE 1

| PATHOLOGIES | SAGITTAL VIEW | AXIAL VIEW |
|---|---|---|
| Pfirmann | Cross-section | / |
| | scanning/volume approach | |
| | with multiple slices | |
| Modic | Cross-section | / |
| | scanning/volume approach | |
| | with multiple slices | |
| Herniated disc | Centered on the herniated | Intervertebral disc, |
| | disc | central |
| Narrow lumbar canal | / | Upper plate spine |
| Foramen | Passing through the centre | / |
| | of the pedicle | |
| Posteriort joints | / | Upper plane spine |
| Spondylolisthesis | Passing through the centre | / |
| | of the vertebra | |
| Vertebral fracture | Cross-section | / |
| | scanning/volume approach | |
| | with multiple slices | |
| Vertebral trays/plates | Cross-section | / |
| | scanning/volume approach | |
| | with multiple slices | |
| Paralumbar muscles: | / | Upper plane spine |
| Psoas/Multifidus/ | | Lower plane spine |
| Erector Spinae | | |
| Spinal body | / | Upper plane spine |
| | | Lower plane spine |

For example, when the diagnose of some conditions like Pfirmann, Modic, Vertebral fracture and vertebral trays/plates use the same types of images as inputs, the first S200 and second S400 processing steps may be combined together, reducing the processing time.

Furthermore, the prediction of a first condition in this list, thanks to the first prediction step S300, may reinforce the probability and reliability of predicting another condition interrelated to the first condition, during the second prediction step S500.

Computer programs implementing the method of the present embodiment can commonly be distributed to users on a distribution computer-readable storage medium such as, but not limited to, an SD card, an external storage device, a microchip, a flash memory device, a portable hard drive and software websites. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this disclosure. All these operations are well-known to those skilled in the art of computer systems.

The instructions or software to control a processor or computer to implement the hardware components and perform the method as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-Res, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

### First System Embodiment

A system 2A according to a first system embodiment of the present disclosure is shown on FIG. 2. The system 2A is configured to provide diagnosis aid for diagnosing spinal conditions and to implement the method previously described.

The system 2A includes an image processing device 4A and a prediction device 6A.

The image processing device 4A (also referred to as “image processing unit”) is configured to process at least one imaging signal associated to a subject, and more specifically one or several imaging signal(s) representative of at least part of the subject's spine, such as one or several image(s) of at least part of the subject's spine. The image processing device 4 is further configured to output spine image data associated to the subject and computed based on said at least one imaging signal. Thus, the image processing device 4A is configured to implement the receiving S100 as well as the S200 first and second S400 processing steps as previously described.

Furthermore, the prediction device 6A (also referred to as “prediction unit”) is configured to compute, based on the spine image data output by the image processing device 4A, at least one feature of a subject's spine. For instance, at least one feature comprises condition data indicative of an occurrence of at least one predetermined condition relating to the subject's spine. Thus, the prediction device 6A is configured to implement the first S300 and second S500 predicting steps as previously described.

According to the present disclosure, each of the expressions “image processing unit” and “prediction unit” should not be construed to be restricted to hardware capable of executing software, and refers in a general way to a processing device, which can for example include a microprocessor, an integrated circuit, or a programmable logic device (PLD). Each of the image processing unit and the prediction unit may also encompass one or more Graphics Processing Units (GPU) or Tensor Processing Units (TSU), whether exploited for computer graphics and image processing or other functions. Furthermore, the expressions “image processing unit” and “prediction unit” should not be construed to be restricted to distinct processing devices. Additionally, the instructions and/or data enabling to perform associated and/or resulting functionalities may be stored on any processor-readable medium such as, e.g., an integrated circuit, a hard disk, a CD (Compact Disc), an optical disc such as a DVD (Digital Versatile Disc), a RAM (Random-Access Memory) or a ROM (Read-Only Memory). Instructions may be notably stored in hardware, software, firmware or in any combination thereof.

### Image Processing Unit 4A

As stated previously, the image processing unit 4A is configured to process at least one input imaging signal associated to the subject in order to compute associated spine image data.

Each imaging signal is, for instance, an X-ray imaging signal, a magnetic resonance imaging (MRI) signal, or an ultrasound signal, such as an X-ray imaging image, a magnetic resonance imaging (MRI) image, or an ultrasound image.

In each of the three aforementioned imaging modalities, the imaging signal may be acquired while the subject is in a static position (static acquisition), or in several successive static positions, for example, flexion and then extension (also referred to as “dynamic acquisition”).

Furthermore, in each of the three aforementioned imaging modalities, the imaging signal may be acquired after injecting a contrast agent in the subject.

For instance, the X-ray imaging signal is acquired using at least one of the following techniques: Computed Tomography, Real Time Radiography, Dual-Energy X-Ray Absorptiometry (DEXA), and/or low-dose biplanar whole-body radiography in functional position (also referred to through its commercial name “EOS”).

For instance, the ultrasound imaging signal is acquired using standard echography and/or ultrasound elastography, the latter being well suited for determining mechanical properties of the imaged tissues.

For instance, the MRI imaging signal is acquired using standard MRI and/or magnetic resonance elastography, the latter being well suited for determining mechanical properties of the imaged tissues.

Preferably, the image processing unit 4A is configured to process imaging signals (more specifically 2D images) acquired according to different acquisition techniques. For instance, in the case of MRI imaging signal, the image processing unit 4A is configured to process imaging signals acquired according to different imaging sequences (T1w, T2w STIR, FLAIR, 3D, Dixon and so on) on at least one of the three anatomical planes (Sagittal, Axial, Coronal).

Hereinafter, said acquired imaging signals are also referred to as “input imaging signals”.

The image processing unit 4A is, for instance, configured to compute the image data by performing, on the input imaging signals, a pre-processing step, an anatomical structure segmentation step, a segmentation post-processing step, a centroid computation step and an intervertebral disc position computation step.

### Pre-Processing

As mentioned above, the image processing unit 4A is preferably configured to apply, during the pre-processing step, at least one predetermined pre-processing transformation to each input imaging signal.

Furthermore, in the case where the input imaging signal is not an image, the image processing unit 4A is configured to compute, during the pre-processing step, an image based on the or each input imaging signal.

For instance, the image processing unit 4A is configured to apply, during the pre-processing step, at least one of: a resizing, an intensity transformation (such as an intensity normalization or an intensity clipping), an artefact correction (such as a bias field correction), a bias correction and a pixel intensity distribution normalization (such as a histogram equalization or a registration method).

In the case of pixel intensity distribution normalization, the image processing unit 4A is, for instance, configured to modify a pixel intensity distribution of the input images to match a reference pixel intensity distribution. Such reference pixel intensity distribution is, preferably, a pixel intensity distribution of images used for training at least one artificial intelligence model implemented by the prediction device 6A, as will be disclosed later.

### Anatomical Structure Segmentation

The image processing unit 4A is also configured to apply, during an anatomical structure segmentation step, a segmentation to each image (pre-processed image if the pre-processing step is performed, and input image if no pre-processing step is performed) in order to identify boundaries of predetermined anatomical structures.

Such anatomical structures include, for instance, vertebrae, intervertebral discs, paraspinal muscles, facet joints, nerves, cysts, osteophytes, edemas and/or blood vessels (artery and vena cava in particular).

For instance, the image processing unit 4A is configured to implement a deep learning model, preferably a 2D convolutional neural network (such as a neural network having a U-Net architecture) to perform such segmentation. In this case, during the anatomical structure segmentation step, each input image (or pre-processed image) is provided to the deep learning model, preferably the convolutional neural network, and, for each image, a set of raw 2D segmentation masks is obtained as an output. Each mask is associated to a corresponding anatomical structure (also referred to as “segmented anatomical structure”) appearing on the image.

An image associated to the corresponding segmentation masks (either raw or post-processed) is also referred to as “segmented image”.

### Segmentation Post-Processing

The image processing unit 4A is also preferably configured to apply, during the post-processing step, at least one predetermined post-processing transformation to each set of raw 2D segmentation masks. Such step is intended, for example, to eliminate false positives and artefacts.

For instance, the image processing unit 4A is configured to perform, during the post-processing step, morpho-mathematical operations (such as erosion, dilatation, closing, opening, watershed transformation and the like) and connected components methods, either on the raw 2D segmentation masks, or on binarized versions thereof. This, for instance, allows to delete connected components of the raw 2D segmentation masks that are smaller than a predetermined threshold, or, in the case of masks corresponding to vertebrae, too far away from the rest of the vertebrae.

In the case of input images corresponding to successive slices of at least part of the subject's spine, representing a 3D view of the at least first and/or second part of the subject's spine, the image processing unit 4A is further configured to compare, as a whole, the successive segmented images, for instance by implementing a voting algorithm. This allows to identify anatomical structures that appear in segmented images corresponding to successive slices and to delete noise and/or artefacts present in said segmented images.

### Centroid Computation

Preferably, in the case where the image processing unit 4A is configured to determine vertebrae segmentation masks, the image processing unit 4A is also configured to compute a vertebra centroid for each segmented vertebra. In this case, for each vertebra, the image processing unit 4A is preferably configured to compute the vertebra centroid as the center of mass of said segmented vertebra, based on the corresponding segmentation masks. In other words, to compute the centroid of a given vertebra, the image processing unit 4A is configured to take into account the segmentation mask corresponding to said vertebra for each image representing the vertebra.

For instance, for a given vertebra, image processing unit 4A is configured to determine a plurality of partial centroids. More precisely, each partial centroid is the geometric center of a section of the vertebra on a respective 2D image representing said vertebra. In this case, the centroid of a given vertebra is the center of mass of all partial centroids corresponding to said vertebra.

The image processing unit 4A is further configured to assign each computed vertebra centroid to the corresponding vertebra.

The image processing unit 4A is advantageously configured to apply a similar processing for determining the centroid of other segmented anatomical structures of the spinal region.

Preferably, the vertebra centroid having the lowest position along a longitudinal axis of the spine is identified as the centroid of the sacrum S1. Alternatively, the centroid of S2 to S5 vertebras could also be identified.

### Intervertebral Disc Position Computation

The image processing unit 4A may be configured to determine a position of intervertebral discs centroids. In this case, the image processing unit 4A is configured to determine, as an intervertebral disc centroid, a point on a spinal curve that is mid-distance between two successive vertebrae centroids.

More precisely, the image processing unit 4A is preferably configured to determine the aforementioned spinal curve. To do so, the image processing unit is advantageously configured to perform a polynomial regression, for instance using the vertebrae centroids as anchor points. The image processing 4A may further be configured to choose the polynomial degree as the number of anchor points.

Alternatively, instead of interpolating the intervertebral discs centroids from the segmented vertebrae, the image processing unit 4A is configured to directly perform segmentation on the intervertebral disc. In this case, the image processing unit 4A is configured to assign, for each intervertebral disc, the corresponding intervertebral disc centroid to the center of mass of said intervertebral disc.

The image processing unit 4A is preferably further configured to compute a bounding box around each intervertebral disc centroid, for instance by using the distance between the intervertebral disc and its neighboring vertebrae to compute height of the bounding box. More generally, for each identified anatomical structure, the corresponding bounding box can be regarded as a region of interest which includes said anatomical structure.

Preferably, the image processing unit 4A is further configured to perform, for each intervertebral centroid, a rotation of the corresponding bounding box using the normal to the spinal curve at the intervertebral centroid. As a result, the bounding boxes all have the same orientation from a segmented image to another, thereby allowing the system 2A to be invariant to relative rotations of the intervertebral discs from a segmented image to another. More generally, for bounding box, the image processing unit 4A may be configured to perform a similar rotation so that, for a given anatomical structure, the corresponding bounding boxes have a same orientation from a segmented image to the other.

Alternatively, the image processing unit is configured to perform object detection methods to directly determine bounding boxes and labels of vertebrae, intervertebral discs and other identified anatomical structures. For instance, in order to determine bounding boxes around the intervertebral discs, the image processing unit 4A is configured to implement a mask region-based deep learning model, preferably a convolutional neural network (R-CNN).

The spine image data output by the image processing unit 4A and received by the prediction unit 6A include, for each anatomical structure, the corresponding determined location (or location of respective landmarks), corresponding boundaries (i.e., outer limits), corresponding bounding box(es), associated measurements (such as distances, areas, volumes and angles within the anatomical structures and between landmarks thereof) and/or corresponding label(s). Preferably, the spine image data preferably also include each image and the corresponding segmentations masks.

For instance, in the case of vertebrae and intervertebral discs, the spine image data computed by the image processing unit 4A include the location of the centroid of each vertebra, the label of each vertebra, the location of the centroid of each intervertebral disc, associated measurements (such as width, height, orientation, etc.) and/or the label of each intervertebral disc, as well as each acquired imaging signal (e.g., each acquired image), associated to the corresponding segmentation masks.

### Prediction Unit 6A

As previously stated, the prediction unit 6A is configured to compute, based on the spine image data provided by the image processing unit 4A, at least one feature of a subject's spine. Each feature includes, for instance, condition data indicative of an occurrence of at least one predetermined condition relating to the subject's spine.

The prediction unit 6A comprises at least a first module 8 (also referred to as “first prediction module”), and a second module 10 (also referred to as “second prediction module”). In the example of FIG. 2, the prediction unit 6A further includes additional modules 12 (also referred to as “third prediction module”) and 14 (also referred to as “fourth prediction module”).

### First Prediction Module 8

The first prediction module 8 is configured to receive a first set of spine image data associated to the subject, among the spine image data output by the image processing unit 4A.

The first prediction module 8 is also configured to compute, based on spine image data representative of at least part of an examined spine, at least one first output relating to a first anatomical structure of the examined spine. Consequently, the first prediction module 8 is configured to compute at least one first output relating to the first anatomical structure of the current patient's spine based on the first set of spine image data. In this case, the at least one first output includes a first feature of the examined spine.

For instance, to compute each first output, the first prediction module 8 is configured to implement an artificial intelligence model.

Furthermore, according to the present disclosure, by “first output of the first module”, it is meant any data or parameter (such as a weight) that is a result of a calculation performed by said first prediction module 8. Such data may be, for example, a grade (number), a landmark (vector), or an area (matrix), a segmentation result, a position, a probability, etc. Furthermore, each parameter may be a weight of the artificial intelligence model, determined either during or after training of said model.

The first feature preferably comprises first condition data (comprised in the aforementioned condition data) indicative of an occurrence of a predetermined first condition in the first anatomical structure.

The first condition (as well as the second condition described hereinafter) is, for instance, one of: Modic type endplate changes, Schmorl node, anterolisthesis, retrolisthesis, laterolisthesis, disc degeneration, hypolordosis, hyperlordosis, scoliosis, disc herniation (symmetric bulging, asymmetric bulging protrusion and extrusion) and its location, sequestration status, nerve root compression status, spinal canal stenosis and its origins, lateral recess stenosis and its origins, neural foraminal stenosis and its origins, compression fracture and its acute status, paraspinal muscle atrophy, fatty involution in paraspinal muscle, facet arthropathy and its origins, tumors, infection, pain, spondylosis and so on.

### Second Prediction Module 10

The second prediction module 10 is configured to receive a second set of spine image data associated to the subject, among the spine image data output by the image processing unit 4A. The second set of spine image data may be identical to or different from the first set of spine image data.

The second prediction module 10 is also configured to receive at least one first output of the first prediction module 8. This is illustrated, on FIG. 1, by an arrow going from the first prediction module 8 to the second prediction module 10.

The second prediction module 10 is further configured to compute, based on spine image data representative of at least part of the examined spine and at least one first output of the first module, at least one second output relating to a second anatomical structure of the examined spine. Consequently, the second prediction module 10 is configured to compute at least one second output relating to the second anatomical structure of the current patient's spine based on the at least one first output and the second set of spine image data. In this case, the at least one second output includes a second feature of the examined spine.

Preferably, the second feature includes second condition data indicative of an occurrence of a predetermined second condition in the second anatomical structure. More precisely, the occurrence of the second condition is related to the occurrence of the first condition. In this case, the second anatomical structure is preferably the first anatomical structure, or a second anatomical structure of the spine, distinct (i.e., different) from the first anatomical structure, for instance an anatomical structure adjacent to the first anatomical structure, or located at a distance from the first anatomical structure.

For instance, to compute each second output, the second prediction module 10 is configured to implement an artificial intelligence model.

As mentioned above, each first output of the first prediction module 8 that is input to the second prediction module 10 may be data or a parameter (such as a weight) computed by said first prediction module 8. Determination of said data may be determined after or during training of the first and second prediction modules 8, 10.

For instance, the second prediction module 10 is configured to compute each second output using at least part of the weights of the model implemented by the first prediction module 8.

Each first output of the first prediction module 8 that is input to the second prediction module 10 may be chosen using a qualitative or a quantitative approach, or a combination of both approaches.

For instance, each first output that is input to the second prediction module 10 may be quantitatively chosen by implementing, during configuration (i.e., training) of the first and second prediction modules 8, 10, an algorithm (such as attention mechanism, gradient descent, and the like) adapted to highlight weight patterns of the model of the first prediction module 8 that are relevant for the model of the second prediction module 10.

As a first example, a first prediction module 8 configured to predict the occurrence of Schmorl nodes and a second prediction module 10 configured to predict the occurrence of Modic type endplate changes are considered. Additionally, a model is trained to determine co-attention coefficients between layer I of the first module 8 and layer j of the second module 10. These co-attention coefficients are representative of which part of the high-level features of layer i of the first prediction module 8 are input to the layer j of the second prediction module 10. This configuration is advantageous because Schmorl nodes and Modic type endplate degeneration share similar indicators like the localization of endplate and hypersignal in T2w imaging. Thus, mid and high-level features can easily be shared through co-attention connections between the Schmorl node prediction module (i.e., the first prediction module 8) and the Modic type endplate changes prediction module (i.e., the second prediction module 10).

Alternatively, each first output that is input to the second prediction module 10 may be qualitatively determined using prior knowledge regarding a relationship between the first feature computed by the first prediction module 8 and the second feature computed by the second prediction module 10.

For instance, in the case where the second condition is disc degeneration, the second prediction module 10 is advantageously configured to receive outputs from a first prediction module 8 configured to compute first condition data indicative of an occurrence of Modic type endplate changes.

In the case where the artificial intelligence model implemented by the first prediction module 8 is a neural network, the choice of each first output that is provided to the second prediction module 10 may relate to a choice of weights at specified depth levels of the neural network. In this case, the weights computed by the first layers of the neural network correspond to information of low complexity, and are referred to as “low level” outputs. Moreover, the weights computed by the last layers of the neural network correspond to information of high complexity, and are referred to as “high level” outputs.

In another example, the second prediction module 10 is configured to take as input, in addition to the second set of spine image data, one or more first feature(s) computed by the first prediction module 8. For example, this configuration is advantageous in the case where the first prediction module 8 is configured to predict the existence of a herniated disc, while the second prediction module 10 is configured to predict the existence of a spinal canal stenosis. Indeed, the presence of a herniated disc is one of the possible origins of spinal canal stenosis. Therefore, the knowledge that there exists a herniated disc is likely to cause the second prediction module 10 to be more reliable.

As another example, the first prediction module 8 is configured to compute first features that include information relating to the occurrence of a given condition, as well as a hint for this condition. In this case, the second prediction module 10 is configured to take as input, in addition to the second set of spine image data, each hint computed by the first prediction module 8. For example, this configuration is advantageous in the case where the second prediction module 10 is configured to predict the existence of a spinal canal stenosis, and the first prediction module 8 is configured to predict the existence of a herniated disc as well as, as a corresponding hint, the localization of the disc and the spinal canal. Since such hint is strongly correlated to the condition that the second prediction module 10 is configured to predict, the reliability of such prediction is highly increased.

As another example, the first prediction module 8 and the second prediction module 10 are associated to anatomical structures that are located at different levels along a direction of the spine, for instance anatomical structures that are located along the direction of the spine, such as adjacent along the direction of the spine. For example, the first prediction module 8 is configured to predict the existence of a spondylolisthesis at a first vertebral level, and the second prediction module 10 is configured to determine the existence of a herniated disc at another vertebral level. This is advantageous, because the spine is a structure along which anatomical structures are mechanically coupled. As a result, a biomechanical property on a given vertebral level (such as spondylolisthesis) is likely to have biomechanical consequences on the neighboring levels. As a result, the reliability of the second prediction module 10 is increased.

Even though the example of FIG. 2 shows one first prediction module 8, and one second prediction module 10, there can be several first prediction modules, and/or several second prediction modules. In this case, at least one pair including a first prediction module and second prediction module as described above is defined. Moreover, a first prediction module may be configured to feed data to several second prediction modules.

### Third Prediction Module 12

As shown on FIG. 2, a double-headed arrow connects the third prediction module 12 and the first prediction module 8. This means that:
on the one hand, the third prediction module 12 is configured to operate as a second prediction module, thereby computing a spine feature (distinct from that computed by the second prediction module 10), based on at least one output of the first prediction module 8 and a third set of image data of the set of image data;
on the other hand, the module 8 is configured to operate as a second prediction module described above, receiving, as inputs, outputs of the third prediction module 12.

Even though the third prediction module 12 is configured to operate as a second prediction module, the third set of image data, the output received from the first prediction module and/or the computed feature differ from those corresponding to the second prediction module 10 described above.

Alternatively, the second prediction module is not provided. In this case, module 8 acts as a first prediction module with respect to module 12, and vice versa.

### Fourth Prediction Module 14

As shown on FIG. 2, the fourth prediction module is configured to compute at least one feature relating to the subject's spine based on at least one acquired imaging signal, without prior image processing. In this case, the corresponding set of image data is the at least one acquired imaging signal.

The fourth prediction module 14 may provide outputs to and/or receive outputs from any of prediction modules 8, 10, 12. For instance, on the example of FIG. 2, the fourth prediction module 14 is configured to provide outputs to and receive outputs from the second prediction module 10.

### Operation

During a configuration step, the first and second prediction modules 8, 10 of the prediction device 6A are configured, and each first output that is shared from the first prediction module 8 to the second prediction module 10 is selected.

In the case where the first and second prediction modules 8, 10 implement artificial intelligence models, said models are trained during the configuration step, either separately or jointly.

Then, during an acquisition step, imaging signals representative of at least part of the spinal region of a subject are acquired.

Then, the image processing device 4A computes the spine image data based on the acquired imaging signals.

Then, during a prediction step, the first prediction module 8 receives a first set of spine image data, and computes at least one corresponding first output. Said at least one first output includes a first feature of the examined spine.

Moreover, during the prediction step, the second prediction module 10 receives at least one first output of the first prediction module 8, and a second set of spine image data, and computes, based thereon, at least one second output. Said at least one second output includes a second feature of the examined spine.

For instance, the first feature and the second feature are displayed to a healthcare provider, on a display or the like.

### EXAMPLE

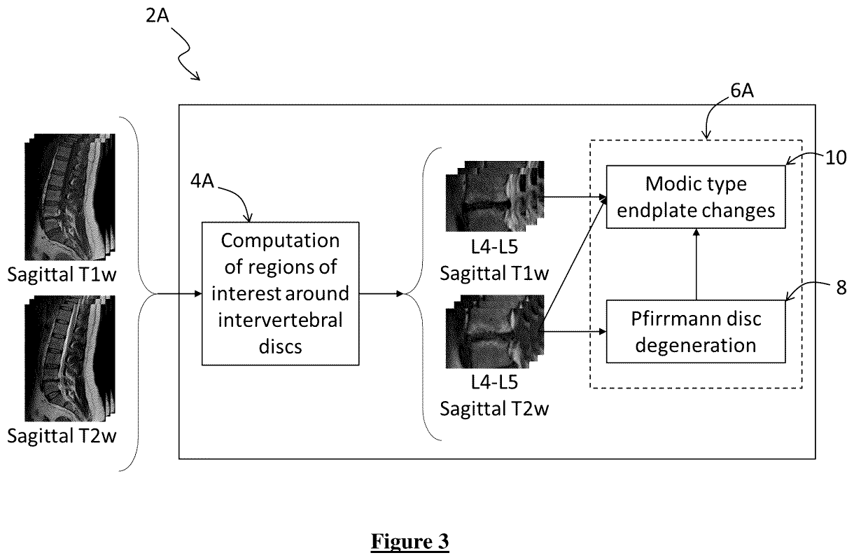

An exemplary implementation of the system 2A is shown on FIG. 3.

In this case, the imaging signals include sagittal sequences T1w and T2w of the lumbar spine of a subject.

These imaging signals are fed to the image processing device 4A. Based on these imaging signals, the image processing device 4A performs pre-processing, segmentation and post-processing steps, and computes regions of interest around the intervertebral discs of the spine, which are provided as an output. For the sake of clarity, only one region of interest around the L4-L5 intervertebral disc is shown on FIG. 3, according to the two original imaging modalities T1w and T2w. These outputs form spine image data.

These regions of interest are provided as inputs to a first modules 8 and a second module 10 which are configured to perform, respectively, diagnosis of disc degeneration and endplate degeneration.

In the case of disc degeneration, the first condition data preferably also comprises a grade on the Pfirrmann scale. Furthermore, in the case of endplate degeneration, the first condition data preferably also includes a grade on the Modic type endplate changes scale. The same applies to the second condition data mentioned below.

Modules 8 and 10 do not have the same inputs. More precisely, the first set of spine image data input to the first module 8 only includes the regions of interest according to the sagittal T2w imaging modality. On the other hand, the second set of spine data input to the second module 10 includes the regions of interest according to sagittal T2w and T1w imaging modalities. Furthermore, the second set of spine data also includes the diagnosis of Pfirrmann disc degeneration performed by the first module 8. This unilateral sharing of information from module 8 to module 10 has been implemented because, from a clinical point of view, the grade on the Pfirrmann scale is an explanatory factor of the grade on the Modic scale.

### Second System Embodiment

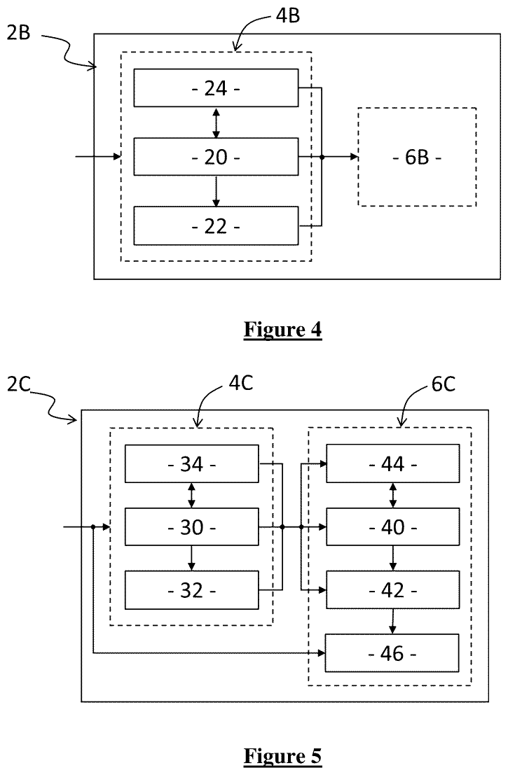

A system 2B according to a second system embodiment of the present disclosure is shown on FIG. 4. Similarly to the system 2A, the system 2B is also configured to provide diagnosis aid of spinal conditions and to implement the method previously described.

The system 2B includes an image processing device 4B, and a prediction device 6B. The image processing device 4B is configured to implement the receiving S100 as well as the S200 first and second S400 processing steps as previously described The prediction device 6B is configured to implement the first S300 and second S500 predicting steps as previously described.

### Image Processing Unit 4B

The second system embodiment differs from the first system embodiment in that the image processing unit 4B includes at least a first module 20 (also referred to as “first image processing module”) and a second module 22 (also referred to as “second image processing module”). In the example of FIG. 4, the image processing unit 4B further includes an additional module 24 (also referred to as “third image processing module”).

### First Image Processing Module 20

The first image processing module 20 is configured to receive a first set of spine image data associated to the subject. In this case, the first set of spine image data corresponds to a set of acquired imaging signals (e.g., images) that are representative of the subject's spine.

Furthermore, the first image processing module 20 is configured to compute, based on spine image data representative of at least part of an examined spine, at least one first output relating to a first anatomical structure of the examined spine. Consequently, the first image processing module 20 is configured to compute at least one first output relating to the first anatomical structure of the current patient's spine based on the first set of spine image data, i.e., the first set of acquired spine imaging signals.

In this case, the at least one first output includes a first feature of the examined spine.

For instance, to compute each first output, the first image processing module 20 is configured to implement an artificial intelligence model.

Furthermore, as stated in relation to the system 2A, a first output may be any data or parameter (such as a weight) that is a result of a calculation performed by said first image processing module 20. Such data may be, for example, a grade (number), a landmark (vector), or an area (matrix), a volume (tensor), a segmentation result, a position, a probability, etc. Furthermore, each weight may be a weight of the artificial intelligence model, determined either during or after training of said model.

### Second Image Processing Module 22

The second image processing module 22 is configured to receive a second set of spine image data associated to the subject. In this case, the second set of spine image data corresponds to a set of acquired imaging signals (e.g., images) that are representative of the subject's spine. The second set of spine image data may be identical to or different from the first set of spine image data.

The second image processing module 22 is also configured to receive at least one first output of the first image processing module 20. This is illustrated, on FIG. 4, by an arrow going from the first image processing module 20 to the second image processing module 22.

Furthermore, the second image processing module 22 is configured to compute, based on spine image data representative of at least part of the examined spine and at least one first output of the first image processing module 20, at least one second output relating to a second anatomical structure of the examined spine. Consequently, the second image processing module 22 is configured to compute at least one second output relating to the second anatomical structure of the current patient's spine based on the at least one first output and the second set of spine image data, i.e., the second set of acquired imaging signals. In this case, the at least one second output includes a second feature of the examined spine.

For instance, to compute each second output, the second image processing module 22 is configured to implement an artificial intelligence model.

Preferably, each of the first feature and the second feature is representative of a geometry of the corresponding anatomical structure. More precisely, the first feature representative of a geometry of a first anatomical structure is related to the second feature representative of a geometry of the second anatomical structure. In this case, the second anatomical structure is preferably the first anatomical structure, or a second anatomical structure of the spine, distinct from the first anatomical structure, for instance an anatomical structure adjacent to the first anatomical structure, or located at a distance from the first anatomical structure.

For instance, for each anatomical structure, the corresponding first feature and/or second feature includes at least one of: corresponding location (or location of respective landmarks), corresponding boundaries (i.e., outer limits), corresponding bounding box(es), associated measurements (such as distances, areas, volumes and angles within the anatomical structures and between landmarks thereof) and/or corresponding label(s).

Advantageously, the first set of spine image data includes at least one imaging signal acquired according to a first acquisition sequence, and the second set of spine image data includes at least one imaging signal acquired according to a second acquisition sequence distinct from the first acquisition sequence. This allows to take into account more information than in the case where only one acquisition sequence is considered, thereby leading to more accurate feature computation. This is especially the case when the second anatomical structure is the first anatomical structure.

In a similar fashion to the configuration of the first and second prediction modules of system 2A, each first output of the first image processing module 20 that is input to the second image processing module 22 may be data or a weight computed by said first image processing module 20.

Furthermore, as discussed previously in relation to the system 2A, each first output of the first image processing module 20 that is input to the second image processing module 22 may be chosen using a qualitative or a quantitative approach, or a combination of both approaches.

As an example, the first image processing module 20 comprises a first stage configured to extract low level features from an acquired imaging signal, and a second stage including a first neural network configured to perform intervertebral disc localization and/or identification. Furthermore, the second image processing module 22 comprises a second neural network configured to perform intervertebral disc segmentation. In this example, the first and second image processing modules 20, 22 are configured so that they share information by having co-attention coefficients between associated layers in the first and second neural networks. These coefficients are learned during training and allow to control how much high-level features from a layer i of the first neural network has to be shared to a layer j of the second module. This is advantageous, especially for segmentation. Indeed, having information regarding the nature of an anatomical structure (for instance: “the detected vertebra is S1”) provides context to the second neural network regarding which vertebrae is being segmenting, thereby allowing the second neural network to perform a more accurate segmentation.

As another example, the first image processing module 20 and the second image processing module 22 are configured to respectively implement a segmentation neural network and a bounding box identification model (also called “region proposal model”). In this case, the region proposal model is configured to perform bounding box identification based on acquired images representing the spine of a subject, as well as high-level features output by the segmentation model. This feature is advantageous, as it speeds up computation and improves the quality of the bounding boxes proposed by the region proposal model.

Even though the example of FIG. 4 shows one first image processing module 20, and one second image processing module 22, there can be several first image processing modules, and/or several second image processing modules. In this case, at least one pair including a first image processing module and second image processing module as described above is defined. Moreover, a first image processing module may be configured to feed data to several second image processing modules.

### Third Image Processing Module 24

As shown on FIG. 2, a double-headed arrow connects the third image processing module 24 and the first image processing module 20. This means that:
on the one hand, the third image processing module 24 is configured to operate as the second image processing module 20, thereby computing a spine feature based at least one output of the first image processing module 20;
on the other hand, the module 20 is configured to operate as a second image processing module described above, receiving, as inputs, outputs of the third image processing module 24.

Alternatively, the second image processing module is not provided. In this case, module 20 acts as a first prediction module with respect to module 24, and vice versa.

### Prediction Unit 6B

The prediction unit 6B is configured to compute, based at least on the first feature and the second feature computed by the image processing unit 4B, condition data indicative of an occurrence of at least one predetermined condition relating to the subject's spine.

The prediction unit 6B is, for instance, configured to implement a known processing pipeline to determine the aforementioned condition data.

### Operation

During a configuration step, the first and second image processing modules 20, 22 of the image processing device 4B are configured, and each first output that is shared from the first image processing module 20 to the second image processing module 20 is selected.

In the case where the first and second image processing modules 20, 22 implement artificial intelligence models, said models are trained during the configuration step, either separately or jointly.

Then, during an acquisition step, imaging signals representative of at least part of the spinal region of a subject are acquired. Said acquired imaging signals form a set of spine image data.

Then, during an image processing step, the first image processing module 20 receives a first set of spine image data of the set of spine image data, and computes at least one corresponding first output. Said at least one first output includes a first feature of the examined spine.

Moreover, during the image processing step, the second image processing module 22 receives at least one first output of the first image processing module 20, and a second set of spine image data of the set of spine image data, and computes, based thereon, at least one second output. Said at least one second output includes a second feature of the examined spine.

Then, during a prediction step, the prediction device 6B computes condition data indicative of an occurrence of at least one predetermined condition relating to the subject's spine based at least one the first and second features computed by the image processing device 4B.

### Third System Embodiment

A system 2C according to a third system embodiment of the present invention is shown on FIG. 5. Similarly to the systems 2A and 2B, the system 2C is also configured to provide diagnosis aid of spinal conditions and to implement the method previously described.

The system 2C includes an image processing device 4C, and a prediction device 6C. The image processing device 4C is configured to implement the receiving S100 as well as the S200 first and second S400 processing steps as previously described The prediction device 6C is configured to implement the first S300 and second S500 predicting steps as previously described.

The image processing device 4C is configured similarly to the image processing device 4B of the system 2B, and includes at least a first module 30 and a second module 32, and preferably a third module 34. More precisely, modules 30, 32 and 34 are similar to modules 20, 22 and 24 respectively of the image processing device 4B of the system 2B.

In this case, the spine image data processed by the image processing device 4C include the acquired imaging signals representative of at least part of the subject's spine.

Furthermore, the prediction device 6C is configured similarly to the prediction device 6B of the system 2B, and includes at least a first module 40 and a second module 42, and preferably a third module 44 and/or a fourth module 46. More precisely, modules 40, 42, 44 and 46 are similar to modules 8, 10, 12 and 14 respectively of the prediction device 6B of the system 2B.

In this case, the spine image data processed by the prediction device 6C include at least first and second features computed by the modules 30, 32 and 34 and, preferably, relating to a geometry of at least one anatomical structure of the subject's spine.

1-20. (canceled)

21. A computer-implemented method for providing diagnosis aid for diagnosing spinal condition in a subject's spine by providing at least one feature of the subject's spine, the method comprising:
receiving at least a first and a second imaging signals associated to the subject, said at least first and/or second imaging signals comprising medical images being representative of at least a part of the subject's spine and/or clinical data relative to said subject;
computing a first set of spine data based on the at least first imaging signal;
predicting at least one first output relating to a first anatomical structure of the subject's spine based on the first set of spine data, the at least one first output including a first feature of the subject's spine including first condition data indicative of an occurrence of a predetermined first condition in the first anatomical structure;
computing a second set of spine data based on the at least second imaging signal; and
predicting at least one second output relating to a second anatomical structure of the subject's spine based on the second set of spine data and on the at least one first output, said at least second output including a second feature of the subject's spine representative of at least one spinal condition.

22. The computer-implemented method as claimed in claim 21, further comprising a pre-processing medical images from the at least first and/or second imaging signals.

23. The computer-implemented method as claimed in claim 22, wherein pre-processing medical images comprises at least one of: a resizing, an intensity transformation, an artefact correction, a bias correction, and/or a pixel intensity distribution normalization.

24. The computer-implemented method as claimed in claim 22, wherein pre-processing medical images is followed by segmenting at least one anatomical structure which processes the images in order to identify boundaries of predetermined anatomical structures.

25. The computer-implemented method as claimed in claim 24, wherein segmenting at least one anatomical structure is implemented by a deep learning model trained on a library of spine images of reference, the deep learning model being configured to output a set of raw 2D segmentation masks, each 2D segmentation mask being associated to a corresponding predetermined anatomical structure appearing respectively in the images pre-processed, said deep learning model being a convolutional neural network.

26. The computer-implemented method as claimed in claim 24, wherein segmenting at least one anatomical structure is followed by:
post-processing on each set of raw 2D segmentation masks so as to eliminate false positives and artefacts, and
labelling the predetermined anatomical structures identified in the segmented images.

27. The computer-implemented method as claimed in claim 24, wherein the predetermined anatomical structures include vertebrae and/or intervertebral discs and, further comprising computing a vertebra centroid for each segmented vertebra and/or computing a intervertebral discs centroid for each segmented intervertebral discs.

28. The computer-implemented method as claimed in claim 27, wherein the vertebra centroid is computed as the center of mass of said segmented vertebra based on the corresponding segmentation masks.

29. The computer-implemented method as claimed in claim 28, further comprising computing an intervertebral disc centroid as the center of mass of said segmented intervertebral disc based on the corresponding segmentation masks.

30. The computer-implemented method as claimed in claim 21, wherein the at least one first output is data or parameter resulting of a calculation performed on the first set of spine data, said at least one first output being at least one of the following such as a grade, a landmark, an area, a volume, a segmented result, a position, a probability, or weights of an artificial intelligence model determined during or after its training for performing the calculation.

31. The computer-implemented method as claimed in claim 21, wherein the first condition is one of: Modic type endplate changes, Schmorl node, anterolisthesis, retrolisthesis, laterolisthesis, disc degeneration, hypolordosis, hyperlordosis, scoliosis, disc herniation (symmetric bulging, asymmetric bulging protrusion and extrusion) and its location, sequestration status, nerve root compression status, spinal canal stenosis and its origins, lateral recess stenosis and its origins, neural foraminal stenosis and its origins, compression fracture and its acute status, paraspinal muscle atrophy, fatty involution in paraspinal muscle, facet arthropathy and its origins, tumors, infection, pain, spondylosis.

32. The computer-implemented method as claimed in claim 21, wherein the second feature includes second condition data indicative of an occurrence of a predetermined second condition in the second anatomical structure, and/or the second condition is related to the occurrence of the first condition.

33. The computer-implemented method as claimed in claim 32, wherein the second anatomical structure is the first anatomical structure.

34. The computer-implemented method as claimed in claim 32, wherein the second anatomical structure is distinct from the first anatomical structure, the second anatomical structure being adjacent to the first anatomical structure or located at a distance from the first anatomical structure.

35. The computer-implemented method as claimed in claim 21, wherein the at least one first output is representative of a relationship between the first feature and the second feature.

36. The computer-implemented method as claimed in claim 35, wherein the first condition data is indicative of an occurrence of Modic type endplate changes and the second condition is disc degeneration.

37. The computer-implemented method as claimed in claim 35, wherein predicting at least one first output and predicting at least one second output are respectively implemented by a first and a second neural networks, the first neural network being trained to generate the at least one first output relating to the first anatomical structure of the subject's spine based on the first set of spine data, and the second neural network being trained to generate the at least one second output relating to the second anatomical structure of the subject's spine based on the second set of spine data and the at least one first output, wherein the at least one first output is related to weights at specific depth levels of the first neural network.

38. The computer-implemented method as claimed in claim 35, wherein predicting at least one first output comprises predicting the existence of a herniated disc, and wherein predicting at least one second output comprises predicting the existence of a spinal canal stenosis.

39. The computer-implemented method as claimed in claim 35, wherein predicting at least one first output comprises computing first features that include information relating to the occurrence of a given condition and a hint for this condition, and wherein predicting at least one second output comprises computing the at least one second output based on the second set of spine data and on the at least one first output comprising the first features and the hint.

40. A system for providing diagnosis aid for diagnosing spinal condition in a subject's spine by providing at least one feature of the subject's spine, the system being configured to implement the method as claimed in claim 21, the system comprising at least one processor configured to:
receive at least a first and a second imaging signals associated to the subject, the at least first and/or second imaging signals comprises medical images being representative of at least a part of the subject's spine; and to compute a first and a second set of spine data respectively based on the at least first and second imaging signals; and
compute at least one first output relating to a first anatomical structure of the subject's spine based on the first set of spine data, the at least first output including a first feature of the subject's spine including first condition data indicative of an occurrence of a predetermined first condition in the first anatomical structure, and to compute at least one second output relating to a second anatomical structure of the subject's spine based on the second set of spine data and on the at least one first output, the at least second output including a second feature of the subject's spine representative of at least one spinal condition.
